(19) United States
(12) Patent Application Publication
(10) Pub. No.: US 2023/0048974 A1
(43) Pub. Date: Feb. 16, 2023

(54) SYSTEMS AND METHODS FOR IMPROVING LIVESTOCK HEALTH AND PERFORMANCE

(71) Applicant: Premex, Inc., Durham, NC (US)

(72) Inventors: Anne BALLOU, Durham, NC (US); Alejandro Mesa GOMEZ, Durham, NC (US); Juan Francisco CHICA JIMÉNEZ, Durham, NC (US); Daniela Valera TABARES, Durham, NC (US)

(21) Appl. No.: 17/884,907

(22) Filed: Aug. 10, 2022

Related U.S. Application Data
(60) Provisional application No. 63/231,320, filed on Aug. 10, 2021.

(51) Int. Cl. G16B 50/30; A01K 11/00; A01K 29/00; G16H 20/10

(52) U.S. Cl. CPC G16B 50/30; A01K 11/006; A01K 29/005; G16H 20/10

(57) ABSTRACT

The present disclosure provides a method of improving health, performance, or a combination thereof for one or more livestock animals is provided. The present systems and methods predict an appropriate intervention for improving animal health, performance, or a combination thereof based on biomarker data.

### CROSS-REFERENCE TO RELATED APPLICATIONS

The present application claims priority to U.S. provisional application number 63/231,320 filed Aug. 10, 2021, the contents of which are incorporated herein.

### BACKGROUND OF THE DISCLOSURE

Gastrointestinal health plays a major role in the safety, efficiency, and profitability of the production of livestock for human consumption. Poor health contributes to food-borne pathogen outbreaks, reduced productivity, and lower economic returns. The causes or contributors of poor gastrointestinal health are complex and gastrointestinal distress can be brought on by a variety of variables and factors.

Currently, the majority of research around gastrointestinal health in livestock is comprised of reductive studies focused on a narrow range of variables. This approach is inherently limited, as such an approach does not mimic or otherwise consider conditions livestock face in production settings and leaves researchers with very little information regarding how many common practices or production systems impact gut health and what impact gut health may have on overall health and performance of the livestock. No known systems are capable of tracking variables at play in gastrointestinal health, gene expression and histology or a combination thereof as a means of predicting changes in the state of health of the gut or other health condition with the goal of improving the overall health and performance of livestock.

### SUMMARY OF THE DISCLOSURE

The present disclosure provides methods of improving health, performance, or a combination thereof for one or more livestock animals. The method includes the steps of:
(a) obtaining a first biomarker data set from one or more livestock animals in need of improvement in health, performance, or a combination thereof, the first biomarker data set including biomarker data obtained from histopathological tissue analysis, gut gene expression analysis, gastrointestinal microbiota analysis, or a combination thereof;
(b) obtaining a historical biomarker data set from a database, wherein the historical data set includes:
   (i) data previously measured from histopathological tissue analysis, gut gene expression analysis, gastrointestinal microbiota analysis, or a combination thereof;
   (ii) data obtained from one or more external databases; or
   (iii) a combination thereof;
(c) processing the first biomarker data set and historical biomarker data set using at least one algorithm executed by at least one processor to identify at least one correlation between the first biomarker data set and historical biomarker data set;
(d) generating at least one suggested intervention predicted to improve health, performance, or a combination thereof;
(e) generating a report comprising the at least one suggested intervention;
(f) transmitting the report to an owner of the livestock, wherein the report is viewable on a livestock owner interface;
(g) confirming or denying acceptance of the report by the livestock owner.

According to one embodiment, upon confirming acceptance of the report, the livestock owner introduces the at least one intervention to the one or more livestock animals. According to one embodiment, the method further includes the step of generating a second biomarker data set from the same or different one or more livestock animals in need of improvement in health, performance, or a combination thereof from which the first biomarker data set was obtained, wherein upon generation of a second biomarker data set, steps (b)-(g) are repeated. According to one embodiment, the histopathological tissue analysis includes the step of: assigning a score to each of one or more of observed traits selected from the group consisting of tissue inflammation severity, lymphoid immunity, microbial organism presence, mucosa integrity, hyperplasia, immune cell infiltration, cell sloughing, necrosis, vascularization, and overall architecture. According to one embodiment, the at least one intervention includes feeding the livestock a customized feedstock recipe, modifying water intake, and administering one or more supplements, medicaments, enzymes, prebiotics, or probiotics to the livestock. According to one embodiment, the customized feedstock recipe includes a change in protein, vitamin, mineral or caloric intake. According to one embodiment, the gut gene expression data is obtained from a livestock host gene selected from the group consisting of interleukins (IL), tumor necrosis factor alpha (TNF-alpha), transforming growth factor beta (TGF-beta), interferon gamma (IFN-gamma), cluster of differentiation (CD) genes, occludin, zonula occludens, claudins, mucin genes, nuclear factor kappa B (NFkB), lipopolysaccharide induced TNF factor (LITAF), toll-like receptors (TLR), secretory IgA (slgA), and beta-defensins. According to one embodiment, the gastrointestinal microbiota analysis includes the step(s) of:
evaluating microbiome diversity including alpha diversity and beta diversity;
performing microbiome differential abundance analysis; or
a combination thereof.

According to one embodiment, the one or more external databases is in wireless communication with one or more data services which aid in and provide third party historical biomarker data related to disease diagnosis, prescriptions medicines, health assessment data, histopathological tissue analysis, gut gene expression analysis, and gastrointestinal microbiota analysis.

### BRIEF DESCRIPTION OF THE DRAWINGS

FIG. 1 is a flow chart illustrating a method for improving health, performance, or a combination thereof for one or more livestock animals according to one embodiment.

FIG. 2 illustrates one embodiment of the system components utilized for improving health, performance, or a combination thereof for one or more livestock animals.

FIG. 3 illustrates one embodiment of the method and system of improving boiler health or performance based on calcium concentration and presence or absence of a vitamin D metabolite in the broiler diet.

FIG. 4 depicts a line graph illustrating the prevalence Operational Taxonomic Units (OTUs) across broiler experimental samples according to one experimental example.

FIG. 5 depicts a dot plot illustrating the differentially abundant sequence analysis associated with a comparison of broilers subject to Vitamin D intervention versus broilers not subject to Vitamin D intervention (over abundant versus under abundant).

FIG. 6 depicts a stacked bar graph illustrating histopathological scores in the cecum aggregated by intervention.

FIG. 7 depicts violin plots illustrating the impact of Vitamin D intervention on IL10 expression in both the cecum and the ileum, where supplementation stimulates opposite effects.

FIG. 8 depicts a dot plot illustrating regression analysis demonstrated between one ratio, termed Ratio 3 (Lactobacillus/rest of the genera), and the expression of MUC2.

### DETAILED DESCRIPTION

The present disclosure will now be described more fully hereinafter with reference to exemplary embodiments thereof. These exemplary embodiments are described so that this disclosure will be thorough and complete, and will fully convey the scope of the disclosure to those skilled in the art. Indeed, the present disclosure may be embodied in many different forms and should not be construed as limited to the embodiments set forth herein; rather, these embodiments are provided so that this disclosure will satisfy applicable legal requirements.

The embodiments described below may assume various alternative orientations and step sequences, except where expressly specified to the contrary. Specific devices and any related processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

As used in the specification, and in the appended claims, the singular forms “a”, “an”, “the”, include plural referents unless the context clearly dictates otherwise.

As used in the specification, and in the appended claims, the term “livestock” refers to one or more animals kept or raised in an agricultural or farm setting for pleasure or profit.

As used in the specification, an in the appended claims, the term “herd” refers to two or more livestock animals of the same species that live together in a social group.

As used in the specification, and in the appended claims, the term “medicament” refers to a substance used for medical treatment.

As used in the specification, and in the appended claims, the term “server” refers to a computer in a network utilized to process and provide data to other computers or components.

As used in the specification, and in the appended claims, the term “database” refers to a data structure or organized collection of electronic data that is electronically accessible.

As used in the specification, and in the appended claims, the term “gut” refers to the digestive track of livestock.

As used in the specification, and in the appended claims, the term “microbiome” refers to the entire habitat of the livestock gut including the genomes of microorganisms, the microorganisms themselves and environment of the livestock gut.

As used in the specification, and in the appended claims, the term “microbiota” refers to bacteria, viruses, fungi, and other microorganisms present in a singular environment, such as the livestock digestive tract.

As used in the specification, and in the appended claims, the term “biomarker” refers to any microbial feature, any gene expression feature, any histopathological feature, or any combination thereof which can relate to, infer, or predict some other trait of interest related to livestock health, livestock performance, livestock productivity, or livestock management.

As used in the specification, and in the appended claims, the term “health” refers to the state (absence or presence) of disease or normal functioning of a livestock animal. Indicators or metrics of health may include, but are not limited to, body temperature; body weight; gait; respirations; herd behavior; coat; skin or feature condition; measured protein, metabolite, or gene expression levels; infection status (parasite, pathogen, etc.) and gut microbiome.

As used in the specification, and in the appended claims, the term “performance” refers to the ability of an animal to exhibit an ecologically relevant task such as running, jumping, feeding, flying, walking, communication, production of a secondary product (e.g. eggs or milk), as well exhibit a weight (e.g., finished weight) and muscle content that is desirable to product a marketable/salable product.

As used in the specification, and in the appended claims, the phrase “improving livestock health and performance” refers to the ability of the systems and methods provided herein to positively impact at least one metric, indicator or task associated with health, performance, or both health and performance.

As used in the specification, and in the appended claims, the term “correlation” refers to a quantifiable relationship between two or more variables. The relationship may be quantified through a variety of statistical and machine learning methods, may have a confidence level associated, and may result in the generation of one or more suggested interventions.

As used in the specification, and in the appended claims, the term “intervention” refers to one or more suggested change or addition related to feedstock recipe, water intake, supplements, medicaments, enzymes, prebiotics, probiotics to the livestock, or any other change or addition deemed fit to improve health, performance, or a combination thereof.

### Overview of Systems and Methods

The present disclosure provides a method of improving health, performance, or a combination hereof for one or more livestock animals. The present systems and methods also have the ability to predict an appropriate intervention for improving animal health, performance or a combination thereof based on a first biomarker data set (e.g., an initial or current biomarker data set) obtained from a target livestock that seeks to have an improvement in health and performance and a historical biomarker data set obtained from previously analyzed livestock (individual livestock animal or normalized over an entire herd of livestock). The present disclosure provides a substantial advancement over the current state of the art which relies on a livestock owner to buy stock feed or other supplements based on limited parameters or data such as livestock weight. These limited parameters are often used as proxy measures for unseen or unmeasured states in the gastrointestinal system (microbiome composition, degree of inflammation or damage), which are not typically obtained on a regular basis. The livestock owner is typically relegated to experimenting with such feed and supplements in hopes of aimlessly finding a combination that improves health and performance. The present disclosure provides an intervention that is customized and predicted to improve health and performance based on biomarker data that is customized and relevant to the livestock seeking such improvements. By improving livestock health, performance, or a combination thereof, a livestock owner is predicted to benefit from improved profits. For example, if the livestock is a herd of beef cattle, the livestock owner’s profits will be improved by having heavier, healthier cattle that will provide more meat at slaughter.

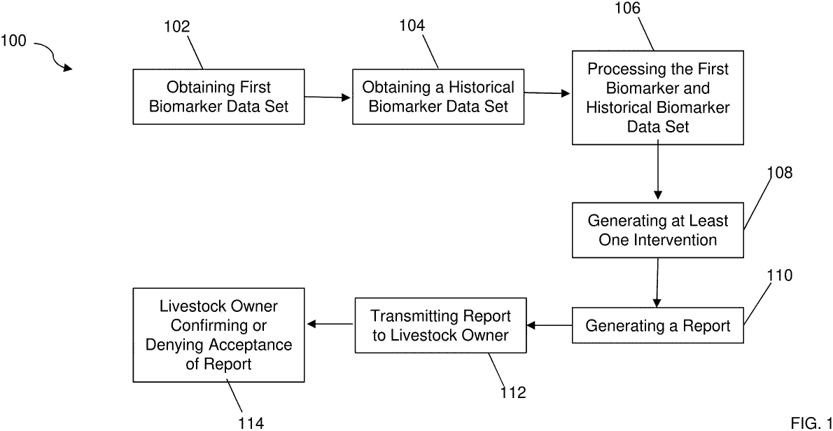

One embodiment of the method 100 of improving health, performance, or a combination hereof for one or more livestock animals is provided in FIG. 1. The method includes the step 102 of obtaining a first biomarker data set from one or more livestock animals in need of improvement in health, performance, or a combination thereof, the biomarker data set including data obtained from histopathological tissue analysis, gut gene expression analysis, gastrointestinal microbiota analysis, or a combination thereof. The method further includes the step 104 of obtaining a historical biomarker data set from a database, wherein the historical data set includes data previously measured from histopathological tissue analysis, gut gene expression analysis, gastrointestinal microbiota analysis, or a combination thereof. The method also includes the step 106 of processing the first biomarker data set and historical biomarker data set using at least one algorithm executed by at least one processor to identify at least one statistically significant correlation between the first biomarker data set and historical biomarker data set.

The method also includes the step 108 of generating at least one intervention predicted to improve health, performance, or a combination thereof. At least one algorithm executed by at least one processor is utilized to identify or otherwise predict at least one intervention.

The method also includes the step 110 of generating a report that includes at least one suggested intervention predicted to improve health, performance, or a combination thereof. The method also includes the step 112 of transmitting the report to an owner of the livestock, wherein the report is viewable on a livestock owner interface. The method also includes the step 114 of confirming or denying acceptance of the report by the livestock owner.

The present systems and methods may also obtain data related to various individual livestock physical and environmental parameters. Such physical and environmental parameters may be provided directly by the livestock owner. Alternatively, the systems may also include sensors for obtaining the physical and environmental data. The physical and environmental data may relate to livestock weight, livestock activity level, livestock ammonia level, body temperature, body weight, water intake, or body pH.

The historical biomarker data set may be maintained in a database for ease of access and continuously updated with data from a variety of sources such as literature or previous biomarker data obtained from similar livestock in a similar geographic region. The historical data set may be utilized to generate a baseline of expected biomarkers on a per-animal, per-farm or per-flock basis thereby allowing for generalizations across geographic areas, between nutritional profiles, and between genetic lines. Both the first biomarker data set and historical biomarker data set include data from histopathological tissue analysis, gut gene expression analysis, gastrointestinal microbiota analysis, or any combination thereof.

The present systems and methods process the first biomarker data set and historical data set using at least one algorithm executed by at least one processor to identify at least one statistically significant correlation between the first biomarker data set and historical data set. Physical and environmental data may also be processed.

By providing a system and method that can assess a livestock environment and various gastrointestinal and physical health parameters of the livestock, a customized report or health and performance improvement plan is generated or otherwise prepared for the livestock owner based on the statistically significant correlation between the first biomarker data set and historical data set. The report sets forth at least one suggested intervention that is predicted to improve the health and performance of the livestock. The intervention is customized and predicted to not only improve gastrointestinal health, but improve overall livestock health and performance. The livestock owner can confirm or approve (and otherwise implement) the at least one intervention set forth in the report.

### System Components

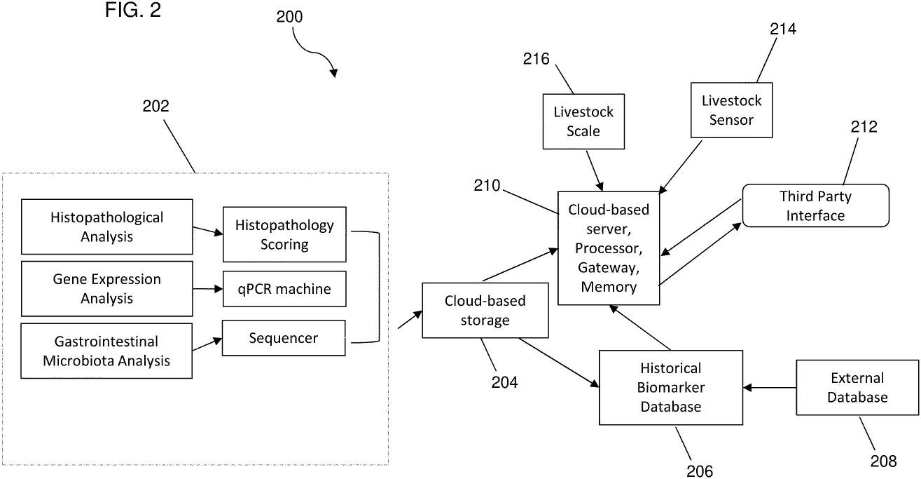

The systems as provided herein includes a variety of components. One embodiment of the system component architecture 200 is set forth in FIG. 2.

Various components 202 of the system 200 may be utilized to obtain or otherwise generate the first biomarker data set. These components 202 include, but are not limited to, a histopathological analysis scoring system, polymerase chain reaction equipment (e.g., qPCR), and sequencing equipment, each of which are described herein.

According to one embodiment, the system 200 includes cloud-based storage 204. The cloud-based storage 204 may include one or more databases such as relational databases. According to one embodiment, the cloud-based storage 204 may receive (e.g., wirelessly) and store the first biomarker data set.

According to one embodiment, a historical marker database 206 is provided that stores and maintains the historical biomarker data sets as provided herein. The historical marker database 206 may store historical biomarker data include data sets that were previously considered first biomarker data as described herein but reclassified as historical biomarker data after initial processing. According to one embodiment, all historical data is received, standardized or normalized and stored for later use. According to one embodiment, all standard data is cleaned and aggregated before further processing.

The historical marker database 206 may maintain one or more licenses to access one or more external databases that, in turn, have licenses or wireless communication access to one or more data services which aid in and provide third party historical biomarker data related to disease diagnosis, prescriptions medicines, health assessment data, histopathological tissue analysis, gut gene expression analysis, and gastrointestinal microbiota analysis. The at least one external database 208 may have licenses, access or storage for third party research and literature-based data that is continuously updated with data obtained and supplied in real-time or substantially real-time. Historical biomarker data may include various standardized data with respect to each of the health profiles of particular livestock types and the genetic information of particular livestock types (herd or individual livestock). According to one embodiment, the historical marker database 206 stores and maintains various historical biomarker data related to previous acquired histopathological tissue analysis, gut gene expression analysis, gastrointestinal microbiota analysis, or a combination thereof, for each of the health profiles of particular livestock types (herd or individual livestock). The historical marker database 206 may be coupled to or in wireless communication with cloud-based storage 204 that is, in turn, in wireless communication with the at least one server, processor, memory, gateway and, optionally, one or more other databases.

The historical marker database 206 may be coupled to or in wireless communication with at least one component 210 that includes at least one server, processor, memory, at least one gateway, or any combination thereof. The at least one component 210 may be part of a computer system. As illustrated, the at least one component 210 includes at least one processor and memory for analyzing the biomarker data sets as provided herein. The cloud-based storage 204 may be in wireless communication with the at least one component 210. Additional data, such as phenotype, performance and environmental data may be fed to introduced to the at least one component 210.

According to one embodiment, the at least one processor described herein utilizes at least one algorithm to identify at least one correlation between the biomarker data set and historical data set. According to one embodiment, one or more algorithms may be utilized to process the data as provided herein to identify at least one correlation. The predicting of at least one intervention may also be carried by at least one processor as provided herein. According to one embodiment, one or more statistical or machine learning models may be utilized by the processor to identify at least one correlation between the first biomarker data set and historical data set. According to one embodiment, the statistical model or machine learning model is one more of linear regression, logistical regression, logarithmical regression, LASSO regression, ridge regression, survival analysis, PERMANOVA, principal component analysis, multidimensional scaling, ARIMA, or any combination thereof. According to one embodiment, the machine learning model is one or more of decision trees, regression trees, random forest, gradient boosting machine, support vector machine, neural network Bayesian network, or any combination thereof. According to one embodiment, a bioinformatics workflow management system may be utilized to compose and execute a series of computation or data manipulation steps. The bioinformatic workflow management system may transform the data from raw sequences to diversity measures and taxonomic information. According to one embodiment, at least one script-based program is utilized to for statistical analysis of the various data described herein. According to one embodiment, at least one python-based script is utilized that includes wraps functions. According to one embodiment, an open source microbiome bioinformatics platform with a plugin architecture may be utilized such as that available from QIIME2. According to one embodiment, at least one R-based script is utilized for statistical computing.

Further processing of the first (or subsequent) biomarker data set and historical biomarker data set may be conducted by statistical models to study the relationship not only between histopathological tissue analysis, gut gene expression analysis, gastrointestinal microbiota analysis data, but also with phenotypic traits of the livestock and environmental data as provided herein. Additionally, with the compilation of data over time, the predictive models may become more achievable and accurate.

According to one embodiment, the system 200 includes a third party interface 212. The third party interface 212 may be utilized by a livestock feed owner/producer. The third party interface 212 may include a data entry system as well as a data visualization platform or cloud-based dashboard. The third party interface may be in wireless communication with the at least one component 210 that includes at least one server, processor, memory, at least one gateway, or any combination thereof.

According to one embodiment, the third party interface 212 may be a customizable livestock owner interface that includes a portal or dashboard for specific access to the system 200 as provided herein. According to one embodiment, the third party interface 212 includes a cloud-hosted dashboard. According to one embodiment, the third party interface 212 includes an application that may be installed on a stationary device such as, for example, a desktop computer. According to one embodiment, the third party interface 212 includes an application that may be installed on a mobile device such as laptop computer or smart device such as a smart phone or tablet. According to any embodiment, a user-friendly dashboard may be provided and embedded within the third party interface 212. According to one embodiment, the third party interface 212 includes at least one means for authentication and authorization. According to one embodiment, each livestock owner utilizes a custom user name and password that allows access to a particular livestock owner via the third party interface 212.

According to one embodiment, the third party interface 212 allows an owner to upload or enter individual livestock input data regarding at least one of feeding method, feed type, feeding schedule, medical history, breed, gender, breeding status, age, and body condition. According to one embodiment, the livestock owner may upload or enter information related to livestock animal stool, hair, blood, saliva, tissue, oral/dental health, skeletal health, general DNA/genetic profile, or gastrointestinal genetic profile, if such information is already known. Any particular livestock animal may be assigned a particular code that is transmitted to identify that particular livestock animal. According to one embodiment, the third party interface 212 provides a questionnaire or a series of questions for the livestock owner to answer.

According to one embodiment, the third party interface 212 allows an owner to review a report generated by at least one component 210 that includes at least one server, processor, memory, at least one gateway, or any combination thereof. The report includes at least one intervention predicted to improve livestock health, performance, or a combination thereof. The report and associated intervention may be accepted or denied by the third party or livestock owner via the third party interface 212. If the report and intervention are accepted, the livestock owner or designated third party agrees to implement the at least one intervention for the livestock seeking an improvement in health, performance, or a combination thereof. Upon acceptance, an acceptance signal may be transmitted back to the at least one component 210 that includes at least one server, processor, memory, at least one gateway, or any combination thereof.

After implementation of the at least one intervention, a second biomarker data set may be obtained from similar livestock of the same owner (or of similar geographic location) processed according to the same methods and with the same system components provided herein with respect to the first biomarker data set. A report may once again be generated that includes at least one intervention for improving livestock health and performance. The livestock owner may once again accept or deny the report. Upon acceptance, an acceptance signal may be transmitted back to the at least one component 210 that includes at least one server, processor, memory, at least one gateway, or any combination thereof. The acceptance signal may cause the first historical data set to be transmitted from the cloud-based storage 204 to the historical biomarker database 206. Any data maintained in the historical biomarker database 206 may be organized in a manner that allows search term indexing and text-based queries.

According to one embodiment, the system 200 may optionally include at least one livestock sensor 214. The at least one livestock sensor may be coupled to or otherwise in communication with at least one component 210 that includes at least one server, processor, memory, at least one gateway, or any combination thereof. According to one embodiment, the at least one sensor 214 is coupled to or wirelessly connected to at least one custom electronic board. According to one embodiment, the electronic board filters a data signal from the sensor and transmits the data signal to the at least one component 210. According to one embodiment, the at least one livestock sensor 214 wirelessly transmits sensor data including data related to at least one of livestock activity level, body temperature, body weight, water intake, body pH, environmental temperature, environmental ammonia level, environmental humidity, rain quantity, wind speed, and trough water temperature. Such sensor data may be optionally be transmitted in real-time.

According to one embodiment, a plurality of sensors 214 is located throughout the agricultural environment or farm where livestock are located. The one or more sensors 214 may be installed in various locations throughout the livestock’s environment. According to one embodiment, the one or more sensors 214 are installed inside a barn or stable. According to one embodiment, the one or more sensors 214 are installed outside a barn or stable such as, for example, in a pasture or grazing area where the livestock reside during daytime hours. According to one embodiment, the at least one sensor 214 detects at least one of livestock activity level, ammonia level in bedding, bedding or cage type, body temperature, body weight, water intake, body pH, environmental temperature, environmental humidity, rain quantity, wind speed, elevation, and trough water temperature. According to one embodiment, certain sensor data is obtained through various commercial sensors. According to one embodiment, a sensor 214 may include or otherwise utilize a statistical-based algorithm to measure livestock physical activity.

According to one embodiment, the system 200 may optionally include at least one livestock scale 216. The livestock scale 216 may be coupled to or otherwise in communication with at least one component 210 that includes at least one server, processor, memory, at least one gateway, or any combination thereof and transmits weight data.

According to one embodiment, any of the data as provided herein may be entered into, received into or maintained in a cloud pipeline system. According to one embodiment, the first biomarker data and historical biomarker data as provided herein may be entered into, received into or maintained in a cloud pipeline system. According to one embodiment, the cloud pipeline system includes at least one server, processor and memory to receive, analyze, process, detect anomalies (if present) and store the data provided herein including the first biomarker data and historical biomarker data. According to one embodiment, the cloud pipeline system includes more than one of a plurality (e.g., cluster) of servers receive, process and store the various data as provided herein. According to one embodiment, the cloud pipeline system includes at least one computer to receive, analyze, process, detect anomalies (if present), and store the various data as provided herein. According to one embodiment, at least one server are configured to receive, process and store the various data as provided herein. The number of servers, computers or a combination thereof is scalable and varies depending on data size.

According to one embodiment, a workflow management system may execute steps that are divided into upstream and downstream analysis. The upstream analysis starts by importing the multiplexed reads (fastq files), demultiplexing the sequences using the barcodes in the sample metadata file (a tab-separated text file), and denoising, the result of which are the amplicon sequence variants (ASVs) or generally called features. In this case the algorithm implemented is DADA2. Downstream analysis includes taxonomic classification and a pipeline to generate the phylogenetic tree. QIIME2 uses scikit-learn (https://scikit-learn.org/stable/) to classify sequences, a pre-trained naive Bayes classifier can be used or a new classifier can be trained as well. The process of creating the three is composed of several steps within the same command: multiple sequence alignment, masking, tree building, and rooting (See https://link.springer.com/protocol/10.1007%2F978-1-4939-8728-3_8).

According to one embodiment, a microbiome bioinformatics platform may be utilized when processing the first (or subsequent) biomarker data set and historical biomarker data set. An example of a suitable microbiome bioinformatics platform includes, but is not limited to, QIIME2. The microbiome bioinformatics platform provides functions to extract diversity measures, create plots and to perform other types of analysis, however, the first biomarker data obtained (feature table, tree, taxonomy) may be exported to be subsequently post processed and analyzed using packages from a programming language such as R/Python for flexibility purposes. Histopathological tissue analysis, gut gene expression analysis, gastrointestinal microbiota analysis data may also be incorporated to generate standard data tables that are to be imported into the interactive dashboard of the whole panel. Multiple visualizations may be generated to explore variable data, including any associated metadata.

### Methods for Improving Health and Performance

A method of improving health, performance, or a combination hereof for one or more livestock animals is provided. According to one embodiment, a livestock owner may own at least one livestock animal that presents a health deficiency, performance deficiency, or a combination thereof. According to one embodiment, a livestock owner may own at least one livestock animal that exhibits room for improvement in health, performance, or a combination thereof. In either of the aforementioned scenarios, the livestock owner may request or be in need of one or more predicted and customized interventions to cause improvement in health, performance, or a combination thereof. According to one embodiment, a custom sampling plan may be initially developed for obtaining the one or more requisite data sets described herein.

### Gastrointestinal Microbiota Analysis

The methods provided herein include the step of obtaining a first biomarker data set from one or more livestock animals in need of improvement in health, performance, or a combination thereof. The first biomarker data set includes data based, at least in part, on data obtained from a step of performing gastrointestinal microbiota analysis.

Gastrointestinal microbiota analysis may include the step of performing bacterial DNA gene sequencing on sequences obtained by initially extracting bacterial DNA from a gastrointestinal or fecal sample. A polymerase chain reaction (PCR) may then be performed on the bacterial DNA isolated from the sample to amplify the bacterial DNA. According to one embodiment, any suitable marker gene may be sequenced. A suitable marker gene includes, for example, 16S rRNA, which is the DNA sequence corresponding to ribosomal RNA or rRNA, a ubiquitous gene in the bacterial kingdom. According to one embodiment, a pair of primers including a forward primer and a reverse primer that complement the V4 region of the 16S rRNA gene among the bacterial DNA sequence may be utilized followed by amplifying the V4 sequence of 16S rRNA gene of the gut microbiota to obtain an amplicon with a length of about 250 bps. The V4 sequence of 16S rRNA gene of the gut microbiota may be purified. The aforementioned steps may be repeated to append synthetic DNA oligomers to the amplicons in order to obtain a library of amplicons with a sequence length of about 315 bp. Next, the size and concentration of the library is measured by an analyzer and fluorometer. Libraries from each sample may be pooled together in equal concentration based on the previous analysis, and the pooled libraries may be added onto a sequencing chip with complementary adaptors on the surface of the sequencing chip. Next, bridge amplification may be performed to amplify fluorescent detecting signals used during the sequencing by synthesis step. Generation of sequence data may be carried out by using polymerases and fluorochrome-labeled nucleotides. As a nucleotide is added to the complementary DNA strand by the polymerase enzyme, the fluorescent marker is recorded, removed, and the process is repeated until the desired number of rounds are complete. A complete sequence of each amplicon may be recorded in this way.

Gastrointestinal microbiota analysis may include evaluation of microbes (bacteria, fungi or viruses) that have colonized or are otherwise present in the intestine of livestock. The microbial population may include both pathogens as well as commensal bacteria. According to a particular embodiment, gastrointestinal microbiota analysis may include evaluation of the type as well as number of specific pathogens, commensal bacteria, nutrients, or minerals in the small intestine, large intestine, colon, hindgut (cecum), or a combination thereof. According to one embodiment, the livestock are sacrificed to allow for collection of intestinal contents (digesta), intestinal tissues, and feces. According to an alternative embodiment, collection of intestinal contents (digesta), intestinal tissues, and feces may be conducted while the livestock are alive. The age of livestock at the time of gastrointestinal microbiota analysis may vary and may be taken into consideration when the various biomarker data sets described herein are processed.

According to one embodiment, gastrointestinal microbiota analysis may include the step of evaluating microbiome diversity including alpha diversity. The alpha diversity analyses may measure the variability of microbiome species within a sample. According to one embodiment, alpha diversity analyses may illustrate microbiome diversity for various gut locations from a single sample or single herd. Larger microbiome alpha diversity may be associated with a positive health status. Alpha diversity analysis may also analyze and map the microbiota in different portions of the gut and evaluate different ranges of richness (number), diversity, or distribution (evenness) therein. Alpha diversity analysis may include evaluation of cecum samples, ileum samples, and fecal samples. According to one embodiment, alpha diversity analyses may identify the types of bacteria present and changes in bacteria present for individual groups of bacteria which may generate changes in the ecosystem and represent differences between applied interventions.

According to one embodiment, gastrointestinal microbiota analysis may include the step of evaluating microbiome diversity including beta diversity. The beta diversity analyses may measure the similarity or dissimilarity of more than one community of livestock. The beta diversity analyses may, more particularly, evaluate differences in bacterial abundances and compositional distance between samples. Compositional distance may be visualized with coordinates analysis (PCoA) with each axis representing a combination of features (ASVs or OTUs) that account for high amounts of variation between samples. Beta diversity analyses may evaluate and illustrate clear differentiation between samples of different gut locations which may be biologically expected since different locations of the gut have different dynamics, roles and environmental conditions.

Gastrointestinal microbiota analysis may include the step of performing microbiome differential abundance analysis. The differential abundance analysis may include carrying out a functional annotation and taxonomic classification, quantifying abundance for a targeted feature and evaluating statistical significance. The differential abundance analysis may be utilized to detect differentially abundant taxa across phenotype groups which aids in characterizing the relationship between a microorganism and the livestock as well as in disease screening. Any microbiome data obtained during analysis can be filtered, merged, and subset to make specific comparisons between any two groups, and at any taxonomic level. The step of differential abundance analysis may be utilized to identify features (i.e., species, OTUs, gene families, etc.) that differ in abundance between two groups of samples according to their particular intervention. These differentially abundant bacteria represent one embodiment of a microbial biomarker. Various methods may be utilized during differential abundance analysis including, but not limited to, edgeR, metagenomeSeq, DESeq/DESeq2, analysis of compositions of microbiomes (ANCOM/ANCOMBC), zero-inflated beta model (ZIBSeq), a zero-inflated generalized Dirichlet-multinomial model (ZIGDM).

Gastrointestinal microbiota analysis data including gastrointestinal microbial population data and DNA sequencing data, may be added or otherwise input into at least one database. The database may be adapted to receive and store gastrointestinal microbial population data including any related bacterial DNA sequencing data as well as data related to gastrointestinal disease. According to one embodiment, the database works with a genomics analyzing software and at least one algorithm executed by at least one processor to identify at least one correlation between the gastrointestinal microbiota analysis data and historical biomarker data set. The correlation from genomics analysis may detect or otherwise identify anomalies, diseases or genetic mutations, as well as variations in bacterial populations. Diseases that may be detected include, but are not limited to, cancer, metabolic disorders, autoimmune disorders, and other related diseases.

### Gene Expression Analysis

The first biomarker data set includes data which may be based, at least in part, on data obtained from a step of performing gut gene expression analysis. The gut gene expression analysis may be utilized to evaluate how one or more changes to a livestock feed or environment impacts levels of messenger RNA (mRNA) arising from gene expression and vice versa. These changes in expression of mRNA are used as an approximation for changes in levels of the proteins they encode. According to one embodiment, gut gene expression analysis may include quantitative polymerase chain reaction (qPCR) to investigate or quantify changes (increases or decreases) in the expression of a particular gene or set of genes by measuring the abundance of a gene-specific transcript.

According to one embodiment, the gut gene expression data is derived from key genes related to various gastrointestinal issues such as inflammation, immune status, and mucosal lining integrity. According to one embodiment, livestock host RNA is isolated or otherwise extracted from tissue samples. These tissue samples may be collected at the time of animal sacrifice and immediately stored in an appropriate environment to maintain RNA integrity for future analysis. An appropriate environment may include storage at less than -40 C, may utilize a liquid preservative whose components preserve and inactivate the RNA in ambient conditions, or a combination of the two. According to one embodiment, the expression of target gastrointestinal genes relative to housekeeping genes (relative quantification) may be evaluated. According to one embodiment, the host gene is one or more interleukins (IL), tumor necrosis factor alpha (TNF-alpha), transforming growth factor beta (TGF-beta), interferon gamma (IFN-gamma), cluster of differentiation (CD) genes, occludin, zonula occludens, claudins, mucin genes, nuclear factor kappa B (NFkB), lipopolysaccharide induced TNF factor (LITAF), toll-like receptors (TLR), secretory IgA (slgA), beta-defensins, or any combination thereof. According to one embodiment, the host gene is one or more of IL-10, IL-1 B, and MUC2.

According to one embodiment, livestock host RNA may be isolated from preserved tissues utilizing guanidinium thiocyanate-based isolation methods, quantified and preserved at less than -40 C for future analysis. According to one embodiment, livestock host RNA may undergo a two-step or one-step quantitation process. For two-step processing, the RNA may first be processed using a reverse transcriptase enzyme to synthesize complimentary DNA (cDNA). This cDNA may then be analyzed using qPCR in an appropriate machine to quantify RNA expression levels of the target gene. According to another embodiment, RNA expression levels of the genes of interest may be quantified in a one-step reverse transcription qPCR analysis (RT-qPCR). Any suitable livestock host gene of interest may be quantified using DNA primers and probes targeting the region of interest. Quantification may be absolute using a standard curve generated from purified target RNA, or relative, in which expression of the target gene is measured relative to the expression of a constitutively expressed endogenous control gene. Examples of an endogenous control gene include but are not limited to GAPDH, 18S rRNA, or ACTB.

According to one embodiment, cDNA generated from a sample of livestock host RNA is added to replicate (2, 3, or 4) wells in a 96-well plate. All samples are normalized such that each well contains the same total mass of RNA/cDNA. All necessary chemical components of the reaction, including suitable primers and probe targeting each gene to be quantified are added at the same concentration to each well. The probes included in the reaction are labeled with fluorophores to allow for fluorometric quantitation of the amplified cDNA targets. According to one embodiment, the fluorophores used include FAM, VIC, HEX, or TAMRA. A thermocycler equipped with an appropriate fluorometer is used to facilitate the PCR reaction that amplifies target gene transcripts and incorporates the fluorescent probe in the resulting amplicons. When sufficient copies of the target gene incorporating the fluorescent probe have been synthesized, the emitted light from these activated probes reaches detectable levels, and this signal is recorded as the cycle threshold (Ct) for the target gene. According to one embodiment, the Ct value of the target gene is normalized to the Ct value of the endogenous control gene in the same well, allowing for comparison of normalized Ct values between wells, samples, or groups of samples. Normalized Ct values can be further transformed and analyzed. One popular transformation includes the delta-delta Ct method, in which relative fold-change in expression between two samples or groups of samples can be shown as 2^-(normalized target group Ct -normalized control group Ct).

According to one embodiment, the expression of livestock host genes may be measured through non-targeted quantitation of RNA transcripts through the use of high-throughput transcriptomic techniques. Following extraction of RNA from livestock host tissues and synthesis of cDNA, additional PCR steps may be conducted to ligate synthetic DNA oligomers to the cDNA, generated libraries of barcoded sequences for each sample. cDNA libraries from multiple samples may be pooled together in equal concentration, and the pooled libraries may be added onto a sequencing chip with complementary adaptors on the surface of the sequencing chip. Next, bridge amplification may be performed to amplify fluorescent detecting signals used during the sequencing by synthesis step. Generation of sequence data may be carried out by using polymerases and fluorochrome-labeled nucleotides. As a nucleotide is added to the complementary DNA strand by the polymerase enzyme, the fluorescent marker is recorded, removed, and the process is repeated until the desired number of rounds are complete. A complete sequence of each amplicon may be recorded in this way. The resulting transcript sequence data can be used to obtain relative abundance data for all measured transcripts. These transcripts may include genes of interest and their isoforms.

According to one embodiment, a workflow management system may execute steps that are divided into upstream and downstream analysis. The upstream analysis starts by importing multiplexed reads or demultiplexed reads (fastq files). If necessary, demultiplexing is accomplished using the barcodes in the sample metadata file (a tab-separated text file). At least one algorithm is implemented to perform sequence alignment to a reference genome. In this case the algorithm implemented is HISAT2. This output may be used to assemble transcripts and generate abundance estimates. One algorithm that may be implemented to generate abundance estimates is Salmon. Sequences are assembled against a current and appropriate reference genome for the livestock host. DESeq2 is used to perform differential abundance analysis and visualization. Further downstream analyses can include weighted co-occurrence analysis (WGCNA) or pathway-based analysis. In one case, a wrapper function such as iDEP may be used to perform several downstream analyses of transcriptomic data.

### Histopathological Tissue Analysis

The first biomarker data set includes data which may be based, at least in part, on data obtained from a step of performing histopathological tissue analysis. The histopathological tissue analysis may be performed on the livestock gastrointestinal lining. According to such an embodiment, segments of intestinal tissue from one or more livestock animal are obtained/harvested and preserved in formaldehyde, processed, and visually examined under magnification by a medical professional (e.g., pathologist). The age of livestock at the time of histopathological tissue analysis may vary and may be taken into consideration when the various biomarker data sets described herein are processed. Histopathological tissue analysis may include an assessment of the incidence and level of various traits including, but are not limited to, incidence and severity of inflammation, tissue hyperplasia, immune cell infiltration, cell sloughing, and other metrics of gastrointestinal integrity and gastrointestinal health. The assessment of the incidence and level of various traits may be recorded as numerical scores. Exemplary numerical scores include those based on a scale of 0 to 5 with higher numbers indicating higher incidence or severity of aberration.

According to one embodiment, one trait assessed during histopathological analysis includes inflammation severity. Such an assessment may consider the presence of and changes in the architecture or integrity of the tissue, such as intestines, due to inflammation. Architectural changes can be chronic, subacute, and acute. Changes that may be assessed include ballooning of the crypt, edema, and loss of crypt cell walls. According to one embodiment, a score may be assigned based on a qualitative or semi-quantitative assessment of the extent of the damage across the various layers of tissue in the intestine. Exemplary numerical scores include those based on a scale of 0 to 5 with higher numbers indicating higher incidence or severity of aberration.

According to one embodiment, one trait assessed during histopathological analysis includes assessment of the level of infiltration of immune cells into the mucosa or serosa of the intestines. The presence of immune cells in the mucosa is normal, but most are in or near aggregations of lymphoid tissue called Gut associated Lymphoid Tissue (GALT). The assessment of higher than normal levels of lymphoid cells throughout the mucosa or serosa indicate a subacute adaptive immune response to a recent or current immune challenge. Similarly, growth in the GALT regions can indicate a pronounced antigenic challenge. According to one embodiment, a score may be assigned based on a qualitative or semi-quantitative assessment of the extent of the infiltration of immune cells into the mucosa or serosa of the intestines. Exemplary numerical scores include those based on a scale of 0 to 5 with higher numbers indicating higher incidence or severity of aberration.

According to one embodiment, one trait assessed during histopathological analysis includes normal and abnormal infiltration or attachment of microorganisms into the mucosa. Analysis of the attachment of microorganisms into the mucosa may include assessment of organism type (i.e. parasite, yeast, or bacteria). Some association of microbes to the apical surface is normal. According to one embodiment, a score may be assigned based on a qualitative or semi-quantitative assessment of infiltration or attachment of microorganisms into the mucosa. Exemplary numerical scores include those based on a scale of 0 to 5 as provided herein with higher scores indicating infiltration of abnormal microbe types and/or excessive levels.

According to one embodiment, one trait assessed during histopathological analysis includes assessment of the uniformity and consistency of mucosal integrity. According to one embodiment, the uniformity and consistency of mucosal integrity at the apical membrane may be assessed. Aberrations include micro-erosion of the microvilli on the apical surface, ulcers, necrosis, and loss of gut-associated lymphoid tissue (GALT). According to one embodiment, a score may be assigned based on a qualitative or semi-quantitative assessment of the uniformity and consistency of mucosal integrity. Exemplary numerical scores include those based on a scale of 0 to 5 with higher numbers indicating higher incidence or severity of aberration.

According to one embodiment, one trait assessed during histopathological analysis includes assessment of morphology and structure of the mucosa. Aberrations would include blunting of villi, loss of mucus-producing goblet cells, and hyperplasia, in which excessive growth of absorptive enterocytes occurs at the expense of other important cell types such as goblet cells and endocrine cells. These aberrations are reparative mechanisms used by the animal, and typically indicate a repeated stress or injury to the gut at this location. According to one embodiment, a score may be assigned based on a qualitative or semi-quantitative assessment of the morphology and structure of the mucosa. Exemplary numerical scores include those based on a scale of 0 to 5 with higher numbers indicating higher incidence or severity of aberration.

According to one embodiment, the step of performing histopathological tissue analysis includes the step of calculating a cumulative score based on the individual histopathological tissue trait scores as described herein. The cumulative score may be utilized as an indicator of overall intestine condition. The cumulative score may also be utilized as the primary indicator of overall histopathological tissue health based on any variety of traits.

### Additional Data

According to one embodiment, additional data provided herein may be transmitted wirelessly to at least one component that includes at least one server, processor, memory, at least one gateway, or any combination thereof. According to one embodiment, additional data provided herein may be processed as part of the first biomarker data set.

According to one embodiment, the additional data includes livestock sensor data, livestock scale data, or a combination thereof. According to one embodiment, additional data that may be processed includes performance data such as feed conversion rate and body weight gain. According to one embodiment, additional data that may be processed includes farm metadata such as the location of livestock (region and/or country).

### Data Processing

The method for improving health and performance of one or more livestock animals further includes the step of processing the first biomarker data set and historical biomarker data set using at least one algorithm executed by at least one processor to identify at least one correlation between the first biomarker data set and historical biomarker data set. The correlation between the first biomarker data set and historical biomarker data set serves a basis for predicting at least one intervention for improving livestock health and performance. The predicting of at least one intervention may also be carried by at least one processor as provided herein.

According to one embodiment, one or more statistical or machine learning models may be utilized by the processor to identify the at least one correlation between the first biomarker data set and historical data set and prediction of at least one intervention useful in improving livestock health and performance. Phenotypic traits of the livestock and environmental data as provided herein may also be included in the processing step.

### Report and Interventions

The method for improving health and performance of one or more livestock animals further includes the step of generating a report that includes at least one suggested intervention predicted to improve health, performance, or a combination thereof. The report may include various identifying information such as a client or user identification number and time stamp.

According to one embodiment, the at least one intervention includes one or more suggested change or addition related to feedstock recipe, water intake, supplements, medicaments, enzymes, prebiotics, probiotics to the livestock, or any other change or addition deemed fit to improve health, performance, or a combination thereof. According to one embodiment, the at least one intervention includes an adaptive and customized nutritional plan to improve gastrointestinal health. The nutritional plan may provide suggestions as to changes in the diet for a particular livestock animal or a group of livestock animals. The changes to diet include, but are not limited to, changes in protein intake and overall caloric intake. According to one embodiment, the at least one intervention includes a customized feedstock recipe that is particularly tailored to the one or more livestock animals. According to one embodiment, the report includes a medical diagnosis and, optionally, at least one prescribed medicament as an intervention.

According to one embodiment, the report includes basic descriptive statistics, an overview of the gastrointestinal data, and areas for improvement or follow up. This report can take the form of any acceptable electronic format (e.g., a PDF or a PowerPoint presentation). Alternatively, the report may be presented in an online dashboard with interactive graphs for data visualization as described herein. The report may be provided on a per-farm basis or even on a per-animal basis.

According to one embodiment, the report includes a medical diagnosis and, optionally, at least one prescribed medicament as an intervention. According to one embodiment, the report provides various information and suggestions for the livestock owner regarding medical disease diagnosis and treatment regimen. According to one embodiment, the report may note any anomalies in the consumption of feed and water as well as anomalies in body temperature that may be associated with disease.

According to one embodiment, the report provides various information and suggestions for the livestock owner regarding market condition information and suggestions for a particular type of livestock. According to one embodiment, the market condition information includes, but is not limited to, market prices, target sale prices, market futures, and other related market information to assist the livestock owner in optimizing the selling logistics. According to one embodiment, the report provides suggestions for changes in farm infrastructure, livestock management practices, or a combination thereof.

According to one embodiment, the report is conveyed to a pre-determined livestock veterinarian to recommend additional interventions such as potential therapeutic components added to a diet/nutritional, genetic, a therapeutic treatment regimen, or a combination thereof. Upon confirmation of acceptance of the report, a wireless signal is sent to a third party such as, for example, a feedstock or medicament producer. Upon acceptance, instructions may be sent to the third party to manufacture and, optionally, ship a feedstock, enzyme, prebiotic, probiotic, or other curative product to the livestock owner in an effort to address the previously detected anomaly upon administration. According to one embodiment, the third party may adjust various nutritional components in feed such as, for example, vitamin content, mineral content, caloric content, and protein content. According to one embodiment, the third party may formulate and manufacture the customized feed for a particular livestock animal or a particular livestock animal group (herd) at a particular farm. The customized feed may then be shipped directly to the livestock owner’s farm for immediate use.

Although specific embodiments of the present disclosure are herein illustrated and described in detail, the disclosure is not limited thereto. The above detailed descriptions are provided as exemplary of the present disclosure and should not be construed as constituting any limitation of the disclosure. Modifications will be obvious to those skilled in the art, and all modifications that do not depart from the spirit of the disclosure are intended to be included with the scope of the appended claims.

### EXPERIMENTAL EXAMPLE 1

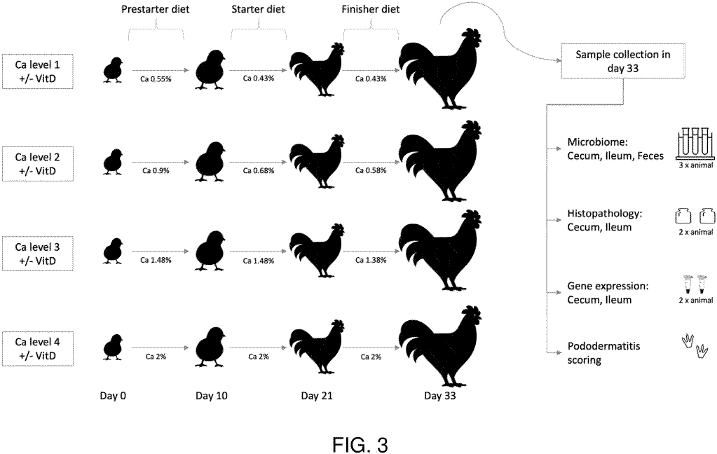

The system and methods provided herein were implemented on an experimental farm located in the town of Fredonia, Antioquia (Colombia) at 1800 meters above sea level with an average annual temperature of 20° C. The system and methods were applied to broilers and evaluated the broilers’ response to interventions in the form of different calcium and vitamin D levels in the broiler’s diet. The experiment consisted of 8 treatments differing in calcium concentration (from deficient to high excess) across the growth stages (see FIG. 3) and also including presence or absence of a vitamin D metabolite in the diet. Histopathological tissue analysis, gut gene expression analysis, and gastrointestinal microbiota analysis data were collected from the broilers at 33 days of age. Samples were collected from six animals per treatment including each gut location (Ileum, cecum, feces as appropriate).

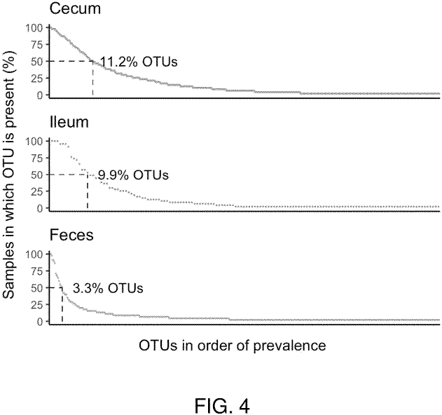

For gastrointestinal microbiota analysis, samples of gut content were collected from three different sampling locations: Cecum, Ileum and Feces. Sequences identified during the sequencing process were grouped in batches of identical sequences so that the number of times that each sequence was found can be recorded. This is the abundance of the sequence. The groups of identical sequences (Operational Taxonomic Units (OTUs) or Amplicon Sequence Variants (ASVs)) often (but not always) correspond to species or genera of bacteria. The prevalence of OTUs across all samples was observed (the fixed threshold for an OTU to be considered present in a sample is 0.1% relative abundance - see FIG. 4). In the present dataset, the sequencing process identified more than 1300 unique sequences, and as expected, only a few OTUs are shared by the majority of samples. In the cecum, for example, only 11.2% of sequences are found in more than half of the samples analyzed. In the feces, this percentage is only 3.3% Most taxa are detected in only a small portion of samples.

Microbiome diversity was then evaluated using established alpha diversity and beta diversity metrics. Alpha diversity showed a few differences between treatment groups within the cecum samples and within the ileum samples. In the fecal samples there were no differences between treatments regardless of the index used. Beta diversity analyzed any differences in bacterial abundances between the samples including compositional distance. The beta analysis found clear differentiation between samples of different gut locations (PERMANOVA evaluation of Bray-Curtis distances, pval=0.001). Statistically significant differences were also found between the treatments within each location, including significance by calcium level and by Vitamin D level.

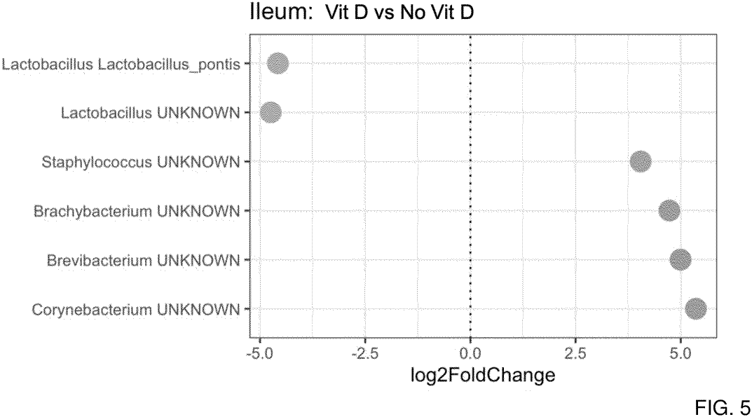

Further methods to identify more specific changes were undertaken. Microbiome data was processed to allow specific comparisons between any two groups, and at any taxonomic level with the goal to identify features (i.e., species, OTUs, gene families, etc.) that differ in abundance between two groups of samples according to their treatment. DESeq was used to identify the features that are differentially abundant between each comparison of two conditions. FIG. 5 demonstrates the differentially abundant sequences associated with one such comparison: samples with Vitamin D vs samples without added Vitamin D. The addition of Vitamin D to the diet resulted in a strong increase in Lactobacillus, and a reduction of similar magnitude in Corynebacterium, a bacteria known to produce ammonia in the litter.

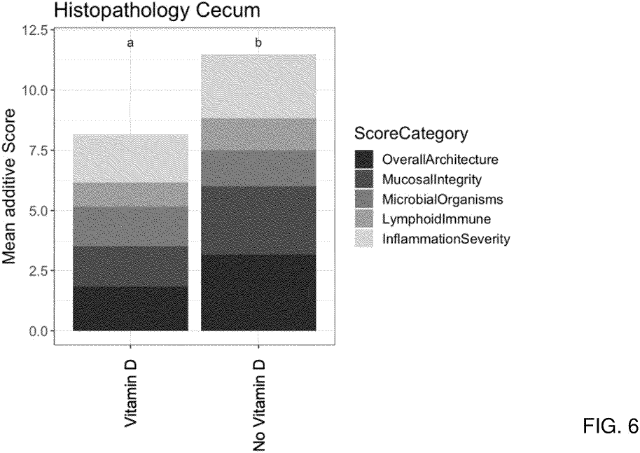

For histopathological analysis, tissue samples were collected from the cecum and ileum. For the histopathological analysis, physical structures of the intestinal tract tissue were analyzed to understand the impact of the intervention of adjusting calcium levels on gastrointestinal inflammation and integrity. A scoring system was employed that allowed for semi-quantitative analysis of the integrity and inflammatory status of the gut. A score of 0 indicated a normal, healthy gut with no appearance of damage or aberration. A score of 5 indicated extreme damage or aberration in the traits being evaluated. The biomarker or traits evaluated included inflammation severity, infiltration of immune cells into the mucosa or serosa, normal/abnormal infiltration or attachment of microorganisms into the mucosa, uniformity and consistency of the mucosa at the apical membrane, and morphology and structure of the mucosa. An individual score for each trait was assigned followed by addition of all individual scores to formulate an additive score. Histopathological scores in the cecum aggregated by intervention are shown in FIG. 6 (cecum). The treatment with recommended intervention levels of calcium with Vitamin D showed better gastrointestinal tissue health compared to the rest of the groups, particularly those with excessive levels of calcium.

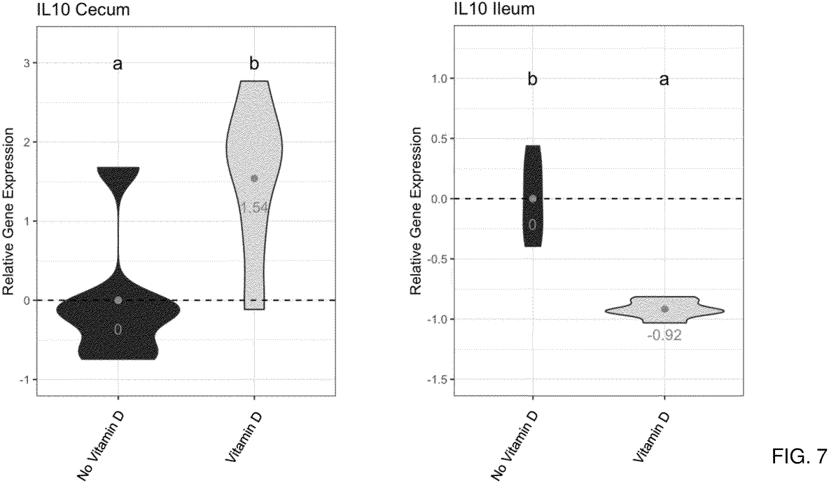

For gut gene expression analysis, tissue samples were collected from the cecum and ileum. Expression for three genes related to inflammation and gut health was quantified, as a way to understand how the gut is responding to the gut microbiota and other stimuli. These target genes were: IL-10, IL-1 B, and MUC2. The resulting data was plotted as the log2 fold-change relative to the control condition (in this case Ca level 2 = control), which has been normalized to a housekeeping gene (GAPDH). The higher the values, the higher the expression of the gene in the intervention treatment(s) compared to the control. Significant differences between groups were tested by ANOVA or multiple t-test (ie Tukey’s test), resulting only in some group differences for the expression of IL10 gene in the cecum, and IL1 B and IL10 genes in the ileum. FIG. 7 demonstrates the impact of Vitamin D on IL10 expression in both the cecum and the ileum, where supplementation stimulates opposite effects. Understanding where in the complex environment of the gut a product or condition exerts an effect represents a rare opportunity in the animal production industry to precisely target and apply treatments for best effect.

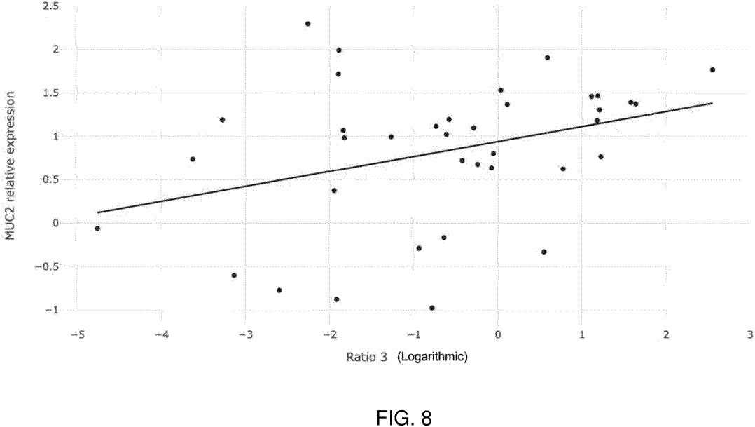

The biomarker data obtained from the histopathological tissue analysis, gut gene expression analysis, and gastrointestinal microbiota analysis was processed using at least one algorithm executed by at least one processor to identify at least one correlation between the obtained biomarker data set and a historical biomarker data set. For example, statistical correlations were generated based on and between the gut gene expression analysis and gastrointestinal microbiota analysis. Linear regressions between single traits (alpha diversity or log ratios of bacteria) and gene expression and histopathology were generated. A regression score was calculated (R-squared), and a line was plotted to show the relationship between the traits. A slope from high to low indicates a negative relationship between the traits, while low-high indicates a positive relationship, and the R-squared value suggests the size of the effect. This regression analysis is demonstrated between one ratio, termed Ratio 3 (Lactobacillus/rest of the genera), and the expression of MUC2 (FIG. 8), with an associated R-squared value of 0.1 suggesting a mild positive association.

Based on the results of these panels and the performance data (not shown) collected during the trial, it was recommended that animals receive Vitamin D supplementation, and that over-inclusion of Calcium in the diet be avoided. Excess calcium strongly and negatively affected cecal mucosal integrity, growth and feed consumption (data not shown) of animals during the study, and this impact was only partially rescued with Vitamin D supplementation. Further, microbial changes in the Vitamin D groups suggest a reduction in ammonia-producing bacteria which could lead to better respiratory and skin health, though measuring these parameters is outside the scope of the current study. Beyond the scope of the current treatments in question, meta-analysis of these and similar datasets allow for the generation of conclusions and hypotheses around how these variables of gut health may correlate to and predict one another. For example, there is a positive relationship between the expression of MUC2 in the ileum and the proportion of Lactobacillus in the population. This and other associations will inform decision makers, product developers, and health care providers in their efforts to promote health and wellbeing in animals in diverse scenarios.

### PROPHETIC EXAMPLE 1

A method improving health, performance, or a combination thereof may be carried out according to the present illustrative example. At the outset, a conversation, questionnaire or a combination thereof may be utilized to obtain initial variable data (including metadata), assuring data linking and define the scope of anticipated data. A test kit may be prepared and utilized in conjunction with each lot of livestock. Each kit may include a unique ID that will be applied to all physical samples and first biomarker data set generated downstream. Additional data related to the date of data collection, exact age of the animals, health status of the flock, and other variables may be collected from the livestock owner.

At the appropriate age (dependent on animal type), livestock animals are selected, euthanized and samples are collected from the following: ileal (small intestine) tissues for histology; ileal tissues for gene expression; ileal contents for microbiome sequencing; cecal (fermentative hindgut region) tissues for histopathological analysis; cecal tissues for gene expression analysis; cecal contents for microbiome analysis; and fecal sample for microbiome analysis. The collection process may be repeated to achieve the desired number of observations for each barn on each farm. The collected samples may be kept stable at room temperature until analysis is conducted (such as during shipping to a lab). The samples may then be processed to enable generation of a first biomarker data set including, but not limited to:
i. Bacterial genomic DNA isolated from fecal and intestinal contents - such contents may be quantified sequence data generated;
ii. Host RNA isolated or extracted from tissue samples, converted into cDNA, and cDNA may be used to quantify the expression of genes of interest using qPCR; and
iii. Formalin-fixed tissue samples may be sent to a service facility for processing, embedding, sectioning, and pathological evaluation.

The first biomarker data set may be entered into cloud-based storage having at least one central database. The database may be utilized to track and index the unique kit ID previously assigned. The first biomarker data set may include, but is not limited to:
i. Summary statistics, including the number of observations and quality of the data;
ii. Statistical analysis and interpretation of gene expression results;
iii. Statistical analysis and interpretation of qualitative and semi-quantitative histology results;
iv. Statistical analysis and interpretation of 16S sequencing results;
v. Statistical analysis and interpretation of the above results in conjunction with one another; and
vi. Interpolative/predictive analysis of flock performance and included health metrics.

The first biomarker data set and historical biomarker data set may then be processed using at least one algorithm executed by at least one processor to identify at least one correlation between the first biomarker data set and historical biomarker data set. At least one suggested intervention predicted to improve health, performance, or a combination thereof may then be generated. The predicted intervention may be generated via at least one algorithm executed by at least one processor and based, at least in part, on at least one correlation between the first biomarker data set and historical biomarker data set.

A report may be generated including the at least one suggested intervention. The report may include recommendations for management and control of gut health based on the above analyses. Such recommendations may include, but are not limited to, alterations in diet composition or feeding strategy, therapeutic intervention, alteration of feed additives used and targeted follow-up analyses.

We claim:

1. A method for improving health, performance, or a combination thereof for one or more livestock animals, the method comprising:
(a) obtaining a first biomarker data set from one or more livestock animals in need of improvement in health, performance, or a combination thereof, the first biomarker data set including biomarker data obtained from histopathological tissue analysis, gut gene expression analysis, gastrointestinal microbiota analysis, or a combination thereof;
(b) obtaining a historical biomarker data set from a database, wherein the historical data set includes:
(i) data previously measured from histopathological tissue analysis, gut gene expression analysis, gastrointestinal microbiota analysis, or a combination thereof;
(ii) data obtained from one or more external databases; or
(iii) a combination thereof;
(c) processing the first biomarker data set and historical biomarker data set using at least one algorithm executed by at least one processor to identify at least one correlation between the first biomarker data set and historical biomarker data set;
(d) generating at least one suggested intervention predicted to improve health, performance, or a combination thereof;
(e) generating a report comprising the at least one suggested intervention;
(f) transmitting the report to an owner of the livestock, wherein the report is viewable on a livestock owner interface;
(g) confirming or denying acceptance of the report by the livestock owner.

2. The method of claim 1, wherein upon confirming acceptance of the report, the livestock owner introduces the at least one intervention to the one or more livestock animals.

3. The method of claim 2, further comprising the step of generating a second biomarker data set from the same or different one or more livestock animals in need of improvement in health, performance, or a combination thereof from which the first biomarker data set was obtained, wherein upon generation of a second biomarker data set, steps (b)-(g) are repeated.

4. The method of claim 1, wherein the histopathological tissue analysis includes the step of:
assigning a score to each of one or more of observed traits selected from the group consisting of tissue inflammation severity, lymphoid immunity, microbial organism presence, mucosa integrity, hyperplasia, immune cell infiltration, cell sloughing, necrosis, vascularization, and overall architecture.

5. The method of claim 1, wherein the at least one intervention includes feeding the livestock a customized feedstock recipe, modifying water intake, and administering one or more supplements, medicaments, enzymes, prebiotics, or probiotics to the livestock.

6. The method of claim 5, wherein the customized feedstock recipe includes a change in protein, vitamin, mineral or caloric intake.

7. The method of claim 1, wherein the gut gene expression data is obtained from a livestock host gene selected from the group consisting of interleukins (IL), tumor necrosis factor alpha (TNF-alpha), transforming growth factor beta (TGF-beta), interferon gamma (IFN-gamma), cluster of differentiation (CD) genes, occludin, zonula occludens, claudins, mucin genes, nuclear factor kappa B (NFkB), lipopolysaccharide induced TNF factor (LITAF), toll-like receptors (TLR), secretory IgA (slgA), and beta-defensins.

8. The method of claim 1, wherein the gastrointestinal microbiota analysis includes the step(s) of:
evaluating microbiome diversity including alpha diversity and beta diversity;
performing microbiome differential abundance analysis; or
a combination thereof.

9. The method of claim 1, wherein the one or more external databases is in wireless communication with one or more data services which aid in and provide third party historical biomarker data related to disease diagnosis, prescriptions medicines, health assessment data, histopathological tissue analysis, gut gene expression analysis, and gastrointestinal microbiota analysis.
